(19) United States
(12) Patent Application Publication
(10) Pub. No.: US 2018/0271429 A1
(43) Pub. Date: Sep. 27, 2018

(54) Allergy Device

(71) Applicants: Kevin Glen, Ventura, CA (US); Patrick Strauss, San Antonio, TX (US); Tom Weisel, Ventura, CA (US); Mark Jennings, San Francisco, CA (US)

(72) Inventors: Kevin Glen, Ventura, CA (US); Patrick Strauss, San Antonio, TX (US); Tom Weisel, Ventura, CA (US); Mark Jennings, San Francisco, CA (US)

(21) Appl. No.: 15/927,982

(22) Filed: Mar. 21, 2018

Related U.S. Application Data
(60) Provisional application No. 62/474,275, filed on Mar. 21, 2017.

(51) Int. Cl. A61B 5/00

(52) U.S. Cl. CPC A61B 5/411; A61B 2560/0223; A61B 2562/08; A61B 2562/247; A61B 2560/0406

(57) ABSTRACT

An allergy testing device is provided. The device may have a protective tray with at least one well. The at least one well is adapted to hold an allergen substance. The device may also have a prong array having at least one prong. The at least one prong may have at least one spike element, which may extend into the at least one well. The device may also have a cover removably engaged with the protective tray to maintain the allergen substance in the well until the device is used to apply the allergen substance to a patient's skin.

### CLAIM OF PRIORITY AND CROSS-REFERENCE TO RELATED APPLICATION

The present application is related to U.S. Provisional Patent Application No. 62/474,275, filed Mar. 21, 2017, entitled “Allergy Device.” The present application hereby claims priority under 35 U.S.C. § 119(e) to U.S. Provisional Patent Application No. 62/474,275.

### BACKGROUND

More than 60 million Americans suffer from allergic diseases, which is the 6th leading cause of chronic disease in the United States. Allergic diseases impose severe restrictions on the physical, psychological and social aspects of patients' lives, and these effects are often under-estimated.

The primary method for testing for allergies in the United States is the skin percutaneous test. A skin test involves a prick with a drop of allergen extract on the surface of the arm or back. This is a well-known technique involving penetration of the allergen into a small area of the skin and then visually observing the reaction, if any, of the skin to the allergen. So far, skin tests have required a complex arrangement of multiple components often coming from different sources stored in different areas of a clinic depending in part on climate control. These typically include: a tray, a well a holder assembly, a well holder assembly lid, a skin prick device with multi-headed pointed ends and biologic allergen extracts in vials to be dispensed into the wells, a pen or marker, and a separate measuring device such as a ruler or caliper.

To perform the test, there are multiple steps. First, the trays must be labelled. Second, individual extracts corresponding to the labels on the trays must be dispensed into the reservoir wells in the well-holder assembly. Both the extracts and the assembly are stored in a refrigerator. These steps are typically only done once for every 40 patients or so. Following these steps, the clinician performing the assay does the following for each patient tested. First, the patient's back or skin is marked in a predetermined sequence, usually A, B, C and so on. Each letter corresponds to the label contained on the trays or otherwise to identify the allergens used for the test. Each label contains an array or battery of typically 8 allergens. After the patient is labelled, the technician typically removes the skin-test device from its packaging and places the device prongs in the wells to adhere a small amount of test solution to the sharp pointed ends, from each test-solution well. The device is removed from the well and applied to a site on the patient's skin in a rocking fashion in accordance with the predetermined sequence corresponding to the labels. The clinician waits approximately 15 minutes, then reviews the reaction on the skin to observe the diameter and height of reactions (if any), typically using a hand-held measuring device such as a ruler or caliper. Because reactions are irregular in shape, the clinician must ensure that diameter is measured at maximum value. The clinician then records the results, manually matching results with the corresponding pattern of the test to ensure accurate patient records.

In the field of allergy testing, the market demands new devices aimed at simplicity and convenience. There is also a significant need for objectivity in allergy test interpretation.

### SUMMARY

At least some embodiments of the invention are directed to a superior and simplified method for percutaneous allergy diagnostics, including a single-use, prefilled percutaneous applicator with unit dose reagents inside a simple ready-to-use pack. The device may also contain a calibrated measuring system in the form of a temporary decal adhered to the patient's skin during the test. The decal may, among other things, identify each allergen being tested and provide a measuring indicator for use in accurately measuring and recording the reaction to the allergen, thus eliminating a major source of error.

In at least some cases, the device decreases the quality problems associated with the need to refill wells and refrigerate trays. This is because, among other things, the devices according to at least some embodiments are self-contained, single-use units.

In at least some cases, the devices also decrease the possibility of contamination, as they are single-use, sealed devices. They may also conserve extract as the blister pack eliminates evaporation and there will be only a drop, or very small portion, used with little or no waste. The extracts will also have superior preservation in the blister containers.

The decal may eliminate human error and provide precision identification of allergens being tested along with precise measurement, for example, of allergen delivery and reaction.

Storage of the devices, at least according to some embodiments, will take much less room than the multi-piece, tray, assembly, extract and device currently used in percutaneous diagnostics. Also, the inventive devices will not require manual assembly and assay performance. Rather, the tests will be stored as flat sleeves of blister packs taking less than 1/10^{th} of the space of conventional components.

According to an example embodiment, an allergy testing device is provided. The device may have a protective tray with at least one well. The at least one well is adapted to hold an allergen substance. The device may also have a prong array having at least one prong. The at least one prong may have at least one spike element, which may extend into the at least one well. The device may also have a cover removably engaged with the protective tray to maintain the allergen substance in the well until the device is used to apply the allergen substance to a patient's skin.

Example embodiments of the invention may include additional components. For example, the allergy testing device may also include a label material disposed between the protective tray and the spacer. The label material may have at least one hole formed therein. The at least one label material hole corresponds to the at least one well. The label material may also have a measuring indicator corresponding to the at least one well. The label material may be adapted to produce a temporary tattoo on a patient's skin.

According to another example, the allergy testing device may also have a cover disposed adjacent the prong array on a side of the prong array opposite the spacer.

According to another example embodiment, an allergy testing device is provided. The allergy testing device has a protective tray having formed therein at least one well. The at least one well is adapted to hold an allergen substance. The allergy testing device also has a prong array having at least one spike element. The at least one spike element extends into the at least one well. The allergy testing device also has a cover removably engaged with the protective tray to maintain the allergen substance in the well until the allergy testing device is used to apply the allergen substance to a patient's skin.

Other example embodiments, features and aspects are discernable from the drawings, the following detailed description, and the claims.

### DESCRIPTION OF THE DRAWINGS

FIG. 1 illustrates a fully-assembled allergen applicator according to an example embodiment;

FIG. 2 is an exploded view of the allergen applicator of FIG. 1;

FIG. 3 illustrates a first step of using the allergen applicator of FIGS. 1 and 2, in a method according to an example embodiment;

FIG. 4 is a rotated view of FIG. 3 viewed from below after the liner has been removed;

FIG. 5 illustrates a second step of using the allergen applicator of FIGS. 1 and 2, in a method according to an example embodiment; and

FIG. 6 illustrates a label component of the devices illustrated in FIGS. 1 and 2.

### DETAILED DESCRIPTION

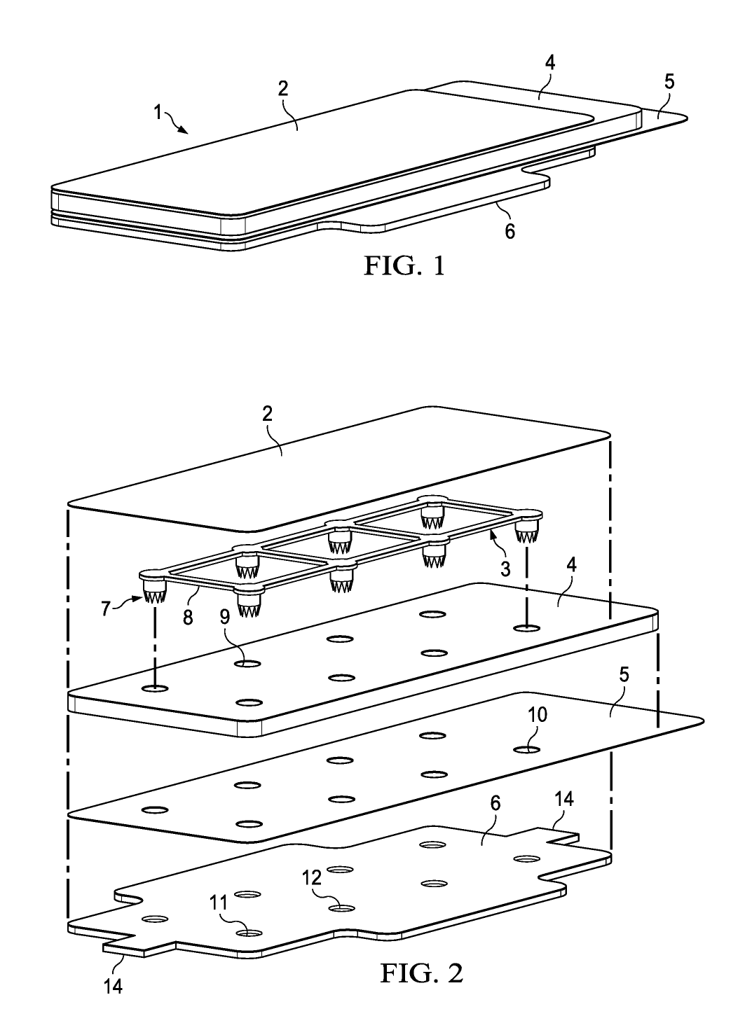

As shown in FIGS. 1 and 2, an allergen applicator device 1 is provided. Device 1 is generally comprised of several layers including protective tray 6, label 5, spacer 4, prong array 3, and cover 2. Generally, in use, it is contemplated that these layers may be separate from one another.

Device 1 includes a protective tray 6, which is on the bottom just below a label 5. Temporarily attached to the label 5 is a spacer 4 with a prong array 3, having spikes 7 extending therefrom. Spikes 7 of the respective prongs are disposed in holes 9 in spacer 4 and the prong array 3 is held in place with a cover 2. The prongs of array 3 are preferably assembled such that each of spikes 7 is disposed in a specific spacer hole 9 and an associated label hole 10 and extend into a well 11 formed in protective tray 6. Each well 11 normally contains a substance 12 that will make contact with a single set of spikes 7.

As an example, the substance 12 may be a liquid and may be a specific allergen. Wells 11 can contain any combination of the same allergen and/or unique allergens. The design is such that when protective tray 6 is removed, the spikes 7 will have a coating of the substance 12 contained in the well 11 in which the respecting spikes 7 were residing. It should be noted that since each layer has adhesive properties between parts that a substance from one well 11 cannot mix with a substance of another well 11. Spikes 7 can then be applied to a patient's skin for a percutaneous allergy test.

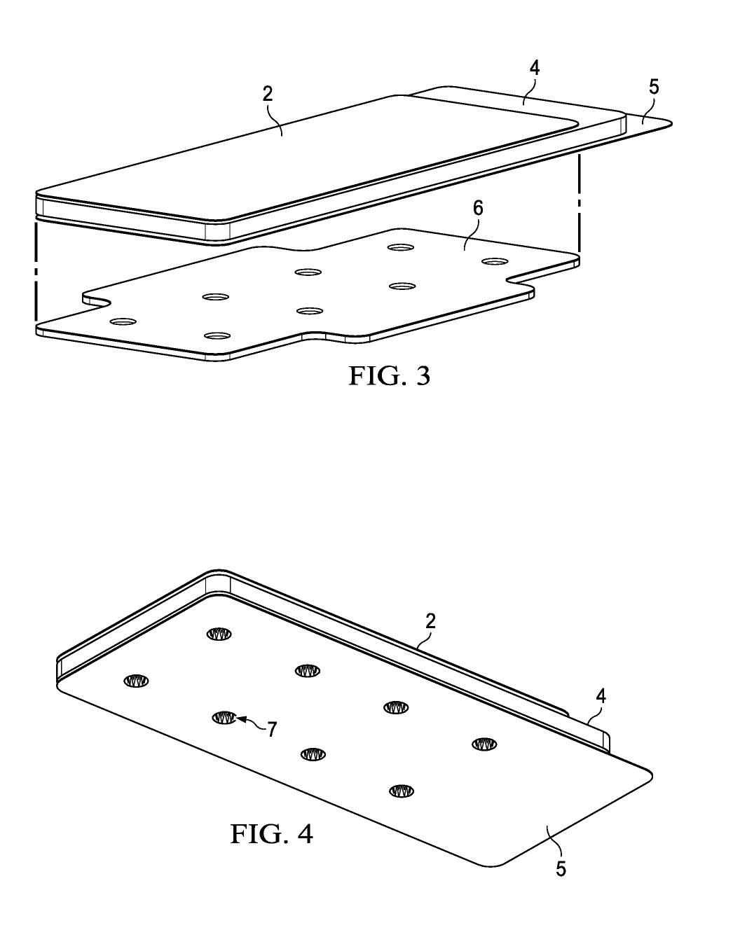

FIG. 3 illustrates a first step of a preferred method of use of the device(s) described in connection with FIGS. 1 and 2. Protective tray 6 is removed from the assembly that also comprises cover 2, spacer 4 and label 5. Prong array 3 is also included in this assembly, but (for convenience) is not expressly shown in FIG. 3. The spikes 7 of the prongs of array 3 are shown in FIG. 4, which is a rotated view of FIG. 3 looking from below. When protective tray 6 is removed, spikes 7 remain coated with the substance 12 that was disposed in the associated well 11.

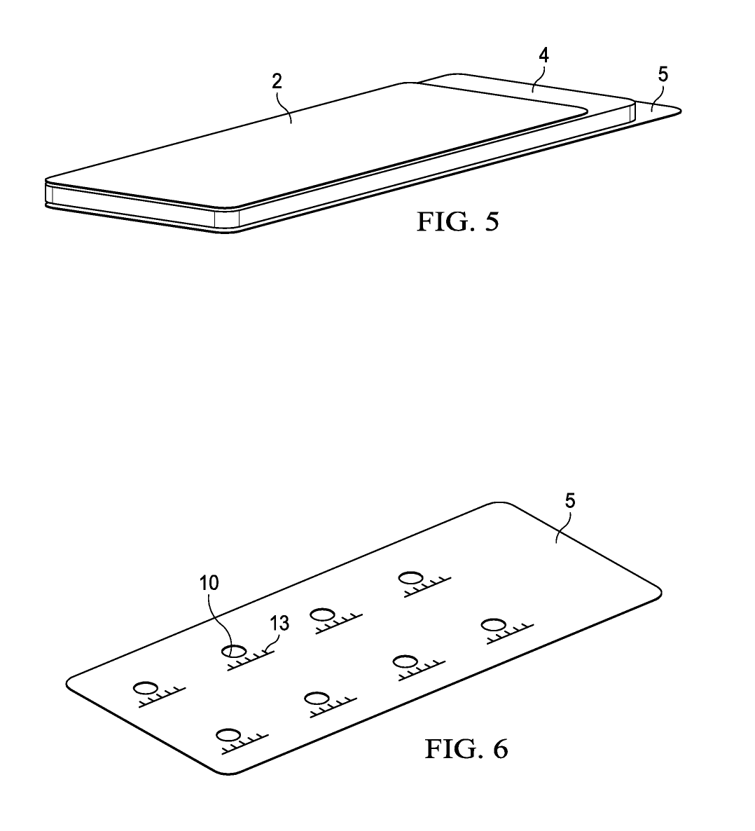

In FIG. 5 the remaining assembly is pressed against the skin of the patient so that the each of the coated spikes 7 delivers and transfers a small amount of the substance 12 to the patient at a set location. After a dwell time, the assembly (including spacer 4, prong array 3 and cover 2) is peeled away leaving the label 5 on the patient's skin. As shown in FIG. 6, the label 5 has markings 13 at each hole 10 that allow the doctor or similar person to easily ascertain the extent of the patient's reaction to each respective substance 12 by presenting a simple size gauge for measurement of the irritation. The label 5 may also include the name of the specific allergen associated with that location. After the reaction measurements are taken the label 5 is peeled off and disposed with the rest of the test kit.

It should be noted that although the size gauge is shown as being linear, other configurations are within the scope of the invention. For example, in one alternative, the size gauge may consistent of a set of concentric circles around a respective hole 10, the circles being of increasing diameter. In another alternative, the gauge may extend across hole 10 itself. Of course, in this alternative, the middle part of the gauge that would otherwise be in the area of hole 10 would not appear. Any suitable size gauge may be incorporated as long as it is associated with a respective hole 10 and allows a user to measure the size of a reaction to an allergen.

In certain embodiments, some of the components of the device may have particular aspects including the following.

The hole on the label 5 may be a larger size than the other holes (of the other components) to make sure it allows for accurate measurement of the magnitude of the reaction. The larger size can also minimize the chance of the label restricting formation of the reaction. One or more radial slits may also be included at each hole to allow for easier deformation.

The label 5 may be of a clear material in order to more easily examine the reaction.

The prongs of array 3 may have connection posts 8 between some or all of the spikes 7 as an aid to plastic flow during molding and minimizing parts during assembly. Also, each spike set can include one spike element or more than one spike element (as shown). Also, the spike elements of the spike set may be inclined inwardly, outwardly, or extend straight down toward the respective well;

The spacer can be made of many different materials including hard bendable plastic or permeable or impermeable foam or gel substrate or a combination thereof. Other suitable materials may be used or included.

Although eight wells and spikes are shown in this example but there can be a device with one or more wells/spikes or a quantity much larger than eight.

The label may be replaced with a temporary tattoo layer that produces temporary tattoos on the patient's skin, each tattoo corresponding to a particular well and/or spike set.

It should be noted that the above described embodiments are examples only and other aspects may be incorporated without deviating from the scope and spirit of the invention. For example, the device is shown as having a rectangular configuration. According to at least one alternative, the device may be round or circular (or of any other shape that allows for the application of allergens to a patient's skin.

According to another example alternative aspect, one or more side tab portions 13 may be included in any of the layers that are removed. The side tab portions allow a user to more easily grasp and remove a respective layer having the side tabs. The side tabs may extend outwardly from one or more edges of the respective layer. Preferably, adjacent layers would not have side tabs that precisely overlap one another as this would hinder a user's ability to grasp a particular side tab.

According to other example embodiment, certain layers may be omitted from the device. For example, according to one example embodiment, the spacer layer is omitted.

According to still other example embodiments, certain layers of the device may be combined. For example, the spacer layer and prong array layer may be combined. That is, the prong array layer may be configured to incorporate its own spacing element such that a separate spacing layer is not needed. In another example, the cover and prong array layer are integrated such that two separate layers are not needed.

As should be apparent to one of ordinary skill in the art, many more aspects and alternatives are well within the scope of the invention, which is defined by the following claims.

1. An allergy testing device, comprising:
a protective tray having formed therein at least one well, said at least one well adapted to hold an allergen substance;
a spacer having formed therein at least one hole, said at least one spacer hole corresponding to said at least one well of said protective tray; and
a prong array disposed adjacent said spacer on a side of said spacer opposite said protective tray, said prong array having at least one prong, said at least one prong having a spike set, said spike set including at least one spike element, said at least one spike set being disposed through said at least one spacer hole and into said at least one well;
wherein, when said protective tray is removed from said allergy testing device, said at least one spike set is adapted to percutaneously apply the allergen substance to a patient's skin.

2. The allergy testing device of claim 1, further comprising:
a label material disposed between said protective tray and said spacer, said label material having at least one hole formed therein, said at least one label material hole corresponding to said at least one well.

3. The allergy testing device of claim 2, wherein said at least one spike set is disposed through said at least one label hole.

4. The allergy testing device of claim 2, wherein the label material is clear.

5. The allergy testing device of claim 2, wherein the label material comprises a measuring indicator corresponding to said at least one well, said measuring indicator enabling a user to measure a patient's skin reaction to the allergen substance.

6. The allergy testing device of claim 5, wherein the measuring indicator is linear.

7. The allergy testing device of claim 5, wherein the measuring indicator is circular.

8. The allergy testing device of claim 2, wherein the label material is adapted to produce a temporary tattoo on a patient's skin, said temporary tattoo corresponding to said at least one well.

9. The allergy testing device of claim 1, further comprising:
a cover disposed adjacent said prong array on a side of said prong array opposite said spacer.

10. The allergy testing device of claim 1, wherein one or more of the cover and the protective tray comprises at least one pull tab extending from a side edge thereof.

11. The allergy testing device of claim 1, wherein said at least one spike element of said at least one spike set is inclined inwardly.

12. The allergy testing device of claim 1, wherein said at least one spike element of said at least one spike set is inclined outwardly.

13. The allergy testing device of claim 1, wherein said at least one well comprises between 4 and 12 wells.

14. The allergy testing device of claim 1, wherein said at least one well comprises between 6 and 10 wells.

15. The allergy testing device of claim 1, wherein the allergy testing device is rectangular in shape.

16. The allergy testing device of claim 1, wherein the allergy testing device is circular in shape.

17. An allergy testing device, comprising:
a protective tray having formed therein at least one well, said at least one well adapted to hold an allergen substance;
a prong array having at least one spike element, wherein said at least one spike element extends into the at least one well; and
a cover removably engaged with the protective tray to maintain the allergen substance in the well until the allergy testing device is used to apply the allergen substance to a patient's skin.

18. An allergy testing device, comprising:
a protective tray having formed therein at least one well, said at least one well adapted to hold an allergen substance;
a prong array having at least one prong, said at least one prong having at least one spike element, wherein said at least one spike element extends into the at least one well;
a spacer having at least one hole formed therein, the spacer disposed between the prong array and the protective tray, the at least one spacer hole corresponding to the at least one well and the at least one prong extending through the at least one spacer hole;
a label disposed between said protective tray and said spacer, said label having at least one hole formed therein, said at least one label hole corresponding to the at least one well and the at least one prong extending through the at least one label hole; and
a cover removably engaged with the protective tray to maintain the allergen substance in the well until the device is used to apply the allergen substance to a patient's skin.
